(19) United States
(12) Patent Application Publication
(10) Pub. No.: US 2026/0083107 A1
(43) Pub. Date: Mar. 26, 2026

(54) Mouse Model For Alzheimer's Disease And Neurofibrillary Pathology

(71) Applicants: United States Government As Represented By The Department Of Veterans Affairs, Washington, DC (US); UNIVERSITY OF MARYLAND, BALTIMORE, Baltimore, MD (US); LOUIS PASTEUR CENTER FOR MEDICAL RESEARCH, Kyoto (JP); The Regents Of The University Of California, Oakland, CA (US)

(72) Inventors: George R. Uhl, Baltimore, MD (US); Yoichiro Iwakura, Kyoto (JP)

(21) Appl. No.: 19/338,694

(22) Filed: Sep. 24, 2025

Related U.S. Application Data
(60) Provisional application No. 63/698,418, filed on Sep. 24, 2024.

(51) Int. Cl. A01K 67/0278; A01K 67/0276; C07K 14/47

(52) U.S. Cl. CPC A01K 67/0278; A01K 67/0276; C07K 14/4702; C07K 14/4711; A01K 2217/072; A01K 2217/075; A01K 2227/105; A01K 2267/0312; A01K 2267/0393

(57) ABSTRACT

Described herein are methods of making and using a transgenic mouse model of Alzheimer's disease. Transgenic animal models and cell lines are disclosed for the study of Alzheimer's disease or Alzheimer's disease-type pathology. Methods of screening and identifying active agents for the treatment of Alzheimer's disease or Alzheimer's disease-type pathology are also provided.

### CROSS REFERENCE TO RELATED APPLICATIONS

This application claims the benefit of the filing date of U.S. Provisional Application No. 63/698,418, which was filed on Sep. 24, 2024. The content of this earlier filed application is hereby incorporated by reference herein in its entirety.

### STATEMENT REGARDING FEDERALLY FUNDED RESEARCH

This invention was made with government support under Grant Numbers DA047713, DA056039, AG058977, AG081210, and AG094378 awarded by the National Institutes of Health. The government has certain rights in the invention.

### BACKGROUND

Alzheimer's disease (AD) pathology includes neurofibrillary tangles (NFTs) rich in hyperphosphorylated tau protein. NFT densities correlate well with the intensity of the cognitive deficits manifest by individuals with AD and can display genetic associations with the receptor type protein tyrosine phosphatase D (PTPRD). Pathological tau hyperphosphorylation is attributed to kinases including the widely-expressed GSK3 α and β.

Activities of GSK3 α and β can be reduced by inhibitors, including Li⁺⁺, and by reduced phosphorylation of their tyrosines pY279 and pY216, respectively. While there are hints that Li⁺⁺ or other systemic GSK3 inhibitors may reduce AD, modest efficacy and substantial toxicities have limited enthusiasm for this approach. Thus, a need exists for new therapies and methods of identifying new therapies for treating AD.

### BRIEF DESCRIPTION OF THE DRAWINGS

FIGS. 1A-C show AT-8 immunoreactive hyperphosphorylated tau in 4 mo 3xTg-AD mice with (L to R) +/+ (FIG. 1A), +/− (FIG. 1B), and −/−PTPRD−/− (FIG. 1C). Inset: 24 mo 3xTg-AD mouse with +/+PTPRD. Bars=100 μm.

FIGS. 2A-B show the neuronal count in 3xTg-AD mice with +/+, +/−, and −/−PTPRD−/− at Bregma −3.80 mm (FIG. 2A) and Bregma −3.08 mm (FIG. 2B).

FIGS. 3A-B show AT-8 immunoreactive hyperphosphorylated tau in 3xTg-AD mice with +/+, +/−, and −/−PTPRD−/− at 4 months, 12 months, and 20 months at Bregma −3.80 mm (FIG. 3A) and Bregma −3.08 mm (FIG. 3B). Bars=100 μm.

FIGS. 4A-H show photomicrographs of hippocampal AT-8 (FIGS. 4A-F) and Aβ (FIGS. 4G-H) immunoreactivities (−3.8 mm from bregma). FIG. 4A shows 4 month-old 3xTg-AD. FIG. 4B shows 20 month-old 3xTg-AD. FIG. 4C shows 4 month-old 3xTg-AD/PTPRD+/−. FIG. 4D shows 4 month-old 3xTg_AD/PTPRD+/−treated with quercetin (25 mg/kg qod ip weeks 6-16). FIG. 4E shows 4 month-old 3xTg-AD mouse treated with pentilludin (100 mg/kg qod po weeks 6-16). FIG. 4F shows 4 month old C57bl (wildtype) mouse treated with pentilludin (100 mg/kg qod po weeks 6-16). FIG. 4G shows 20 month old 3xTg-AD. FIG. 4H shows 20 month old 3xTg-AD/PTPRD+/−. Bar 100 μM.

FIGS. 5A-B show immunoreactivity in neurons after altering PTPRD. FIG. 5A shows the numbers of hippocampal neurons with AT8-immunoreactive hyperphosphorylated tau (+/−SEM) averaged in half brain sections from −3.08 and −3.8 mm from bregma in mice with ages, genotypes and pharmacological treatments indicated. FIG. 5B shows the numbers (+/−SEM) of hippocampal and cortical Aβ immunoreactive elements in half brain sections from −3.8 mm from bregma in 20 month-old mice with genotypes indicated.

FIGS. 6A-B show AT8 immunoreactivity in hippocampal neurons after treatment with vehicle, quercetin, or 6BrQ. FIG. 6A shows the numbers of hippocampal neurons that contain AT8-immunoreactive hyperphosphorylated tau in sections from brains of mice sacrificed at 16 weeks of age which were treated from weeks 6 to 16 of their lives with every other day intraperitoneal 25 mg/kg doses of vehicle, quercetin, or 6BrQ, as indicated. FIG. 6B shows photomicrographs of AT8 immunoreactivity in sections of hippocampus of representative mice treated with vehicle, quercetin or 6BrQ and sacrificed at 16 weeks of age.

### SUMMARY

Disclosed herein is transgenic mouse whose genome comprises: a) a knock-in first transgene encoding a mutant human presenilin-1 polypeptide having a mutation associated with Alzheimer's disease (AD) or AD-type pathology; b) a second transgene, encoding mutant human Tau protein having a mutation associated with AD or AD-type pathology, wherein the second transgene is operably linked to a promoter; and c) a third transgene, encoding human β-amyloid precursor protein (βAPP) having a mutation associated with AD or AD-type pathology, wherein the transgene is operably linked to a promoter; wherein at least one allele of the receptor type protein tyrosine phosphatase D (PTPRD) gene in the genome is ablated or deleted; and wherein expression of the transgenes results in a Tau or Aβ pathology in the transgenic mouse.

### DETAILED DESCRIPTION

The present disclosure can be understood more readily by reference to the following detailed description of the invention, the figures and the examples included herein.

Before the present methods and compositions are disclosed and described, it is to be understood that they are not limited to specific synthetic methods unless otherwise specified, or to particular reagents unless otherwise specified, as such may, of course, vary. It is also to be understood that the terminology used herein is for the purpose of describing particular aspects only and is not intended to be limiting. Although any methods and materials similar or equivalent to those described herein can be used in the practice or testing of the present invention, example methods and materials are now described.

Moreover, it is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including matters of logic with respect to arrangement of steps or operational flow, plain meaning derived from grammatical organization or punctuation, and the number or type of aspects described in the specification.

All publications mentioned herein are incorporated herein by reference to disclose and describe the methods and/or materials in connection with which the publications are cited. The publications discussed herein are provided solely for their disclosure prior to the filing date of the present application. Nothing herein is to be construed as an admission that the present invention is not entitled to antedate such publication by virtue of prior invention. Further, the dates of publication provided herein can be different from the actual publication dates, which can require independent confirmation.

### DEFINITIONS

As used in the specification and the appended claims, the singular forms “a,” “an” and “the”include plural referents unless the context clearly dictates otherwise.

The word “or” as used herein means any one member of a particular list and also includes any combination of members of that list.

Ranges can be expressed herein as from “about” or “approximately” one particular value, and/or to “about” or “approximately” another particular value. When such a range is expressed, a further aspect includes from the one particular value and/or to the other particular value.

Similarly, when values are expressed as approximations, by use of the antecedent “about,” or “approximately,” it will be understood that the particular value forms a further aspect. It will be further understood that the endpoints of each of the ranges are significant both in relation to the other endpoint and independently of the other endpoint. It is also understood that there are a number of values disclosed herein and that each value is also herein disclosed as “about” that particular value in addition to the value itself. For example, if the value “10” is disclosed, then “about 10” is also disclosed. It is also understood that each unit between two particular units is also disclosed. For example, if 10 and 15 are disclosed, then 11, 12, 13, and 14 are also disclosed.

As used herein, the terms “optional” or “optionally” mean that the subsequently described event or circumstance may or may not occur and that the description includes instances where said event or circumstance occurs and instances where it does not.

As used herein, the term “sample” is meant a tissue or organ from a subject; a cell (either within a subject, taken directly from a subject, or a cell maintained in culture or from a cultured cell line); a cell lysate (or lysate fraction) or cell extract; or a solution containing one or more molecules derived from a cell or cellular material (e.g. a polypeptide or nucleic acid), which is assayed as described herein. A sample may also be any body fluid or excretion (for example, but not limited to, blood, urine, stool, saliva, tears, bile) that contains cells or cell components.

As used herein, the term “subject”refers to the target of administration, e.g., a human. Thus, the subject of the disclosed methods can be a vertebrate, such as a mammal, a fish, a bird, a reptile, or an amphibian. The term “subject” also includes domesticated animals (e.g., cats, dogs, etc.), livestock (e.g., cattle, horses, pigs, sheep, goats, etc.), and laboratory animals (e.g., mouse, rabbit, rat, guinea pig, fruit fly, etc.). In one aspect, a subject is a mammal. In another aspect, a subject is a human. The term does not denote a particular age or sex. Thus, adult, child, adolescent and newborn subjects, as well as fetuses, whether male or female, are intended to be covered.

As used herein, the term “comprising” can include the aspects “consisting of” and “consisting essentially of. ” “Comprising” can also mean “including but not limited to. ” “Inhibit,” “inhibiting” and “inhibition” mean to diminish or decrease an activity, response, condition, disease, or other biological parameter. This can include, but is not limited to, the complete ablation of the activity, response, condition, or disease. This may also include, for example, a 10% inhibition or reduction in the activity, response, condition, or disease as compared to the native or control level. Thus, in an aspect, the inhibition or reduction can be a 10, 20, 30, 40, 50, 60, 70, 80, 90, 100%, or any amount of reduction in between as compared to native or control levels. In an aspect, the inhibition or reduction is 10-20, 20-30, 30-40, 40-50, 50-60, 60-70, 70-80, 80-90, or 90-100% as compared to native or control levels. In an aspect, the inhibition or reduction is 0-25, 25-50, 50-75, or 75-100% as compared to native or control levels. “Modulate”, “modulating” and “modulation” as used herein mean a change in activity or function or number. The change may be an increase or a decrease, an enhancement or an inhibition of the activity, function or number.

The terms “alter” or “modulate” can be used interchangeable herein referring, for example, to the expression of a nucleotide sequence in a cell means that the level of expression of the nucleotide sequence in a cell after applying a method as described herein is different from its expression in the cell before applying the method.

“Promote,” “promotion,” and “promoting” refer to an increase in an activity, response, condition, disease, or other biological parameter. This can include but is not limited to the initiation of the activity, response, condition, or disease. This may also include, for example, a 10% increase in the activity, response, condition, or disease as compared to the native or control level. Thus, in an aspect, the increase or promotion can be a 10, 20, 30, 40, 50, 60, 70, 80, 90, 100%, or more, or any amount of promotion in between compared to native or control levels. In an aspect, the increase or promotion is 10-20, 20-30, 30-40, 40-50, 50-60, 60-70, 70-80, 80-90, or 90-100% as compared to native or control levels. In an aspect, the increase or promotion is 0-25, 25-50, 50-75, or 75-100%, or more, such as 200, 300, 500, or 1000% more as compared to native or control levels. In an aspect, the increase or promotion can be greater than 100 percent as compared to native or control levels, such as 100, 150, 200, 250, 300, 350, 400, 450, 500% or more as compared to the native or control levels.

As used herein, the term “determining” can refer to measuring or ascertaining a quantity or an amount or a change in activity.

The term “vector” or “construct” refers to a nucleic acid sequence capable of transporting into a cell another nucleic acid to which the vector sequence has been linked. The term “expression vector” includes any vector, (e.g., a plasmid, cosmid or phage chromosome) containing a gene construct in a form suitable for expression by a cell (e.g., linked to a transcriptional control element). “Plasmid” and “vector” are used interchangeably, as a plasmid is a commonly used form of vector. Moreover, the invention is intended to include other vectors which serve equivalent functions.

The term “expression vector” is herein to refer to vectors that are capable of directing the expression of genes to which they are operatively-linked. Common expression vectors of utility in recombinant DNA techniques are often in the form of plasmids. Recombinant expression vectors can comprise a nucleic acid as disclosed herein in a form suitable for expression of the acid in a host cell. In other words, the recombinant expression vectors can include one or more regulatory elements or promoters, which can be selected based on the host cells used for expression that is operatively linked to the nucleic acid sequence to be expressed.

The term “sequence of interest” or “gene of interest” can also mean a nucleic acid sequence, that is partly or entirely homologous to an endogenous gene of the cell into which it is introduced, but which is designed to be inserted into the genome of the cell in such a way as to alter the genome (e.g., it is inserted at a location which differs from that of the natural gene or its insertion results in “a knockout”). For example, a sequence of interest can be cDNA, DNA, or mRNA.

The term “operatively linked to” refers to the functional relationship of a nucleic acid with another nucleic acid sequence. Promoters, enhancers, transcriptional and translational stop sites, and other signal sequences are examples of nucleic acid sequences operatively linked to other sequences. For example, operative linkage of DNA to a transcriptional control element refers to the physical and functional relationship between the DNA and promoter such that the transcription of such DNA is initiated from the promoter by an RNA polymerase that specifically recognizes, binds to and transcribes the DNA.

As used herein, the terms “promoter,” “promoter element,” or “promoter sequence” are equivalents and as used herein, refers to a DNA sequence which when operatively linked to a nucleotide sequence of interest is capable of controlling the transcription of the nucleotide sequence of interest into mRNA. A promoter is typically, though not necessarily, located 5′ (i.e., upstream) of a nucleotide sequence of interest (e.g., proximal to the transcriptional start site of a structural gene) whose transcription into mRNA it controls, and provides a site for specific binding by RNA polymerase and other transcription factors for initiation of transcription.

Suitable promoters can be derived from genes of the host cells where expression should occur or from pathogens for this host cells (e.g., tissue promoters or pathogens like viruses). If a promoter is an inducible promoter, then the rate of transcription increases in response to an inducing agent. In contrast, the rate of transcription is not regulated by an inducing agent if the promoter is a constitutive promoter. Also, the promoter may be regulated in a tissue-specific or tissue preferred manner such that it is only active in transcribing the associated coding region in a specific tissue type(s). The term “tissue specific” as it applies to a promoter refers to a promoter that is capable of directing selective expression of a nucleotide sequence or gene of interest to a specific type of tissue in the relative absence of expression of the same nucleotide sequence or gene of interest in a different type of tissue.

The phrase “at least” preceding a series of elements is to be understood to refer to every element in the series. For example, “at least one”includes one, two, three, four or more.

As used herein, the term “transgene” describes genetic material that has been or will be or is about to be inserted into the genome of a cell (e.g., a mammalian cells for implantation into a living animal).

As used herein, the term “transformation” refers to a permanent or transient genetic change induced in a cell following incorporation of exogenous DNA to the cell.

As used herein, the phrase “transgenic animal” refers to a non-human animal, generally, a mammal (e.g., mouse, rat, rabbit, etc.) having a non-endogenous (e.g., heterologous) nucleic acid sequence present as an extrachromosomal element in a portion of its cell or stably integrated into its germ line DNA (e.g., in the genomic sequence of most or all of its cells). The phrase “transgenic animal” also includes the founder transgenic non-human animal and progeny of the founders as well as cells, cell lines and tissues from such animals in which one or more of the cells of the animal includes one or more transgenes.

As used herein, “knock-out” of a gene means an alteration in the sequence of the gene or sequence associated with the gene that results in a decrease of function of the target gene. For example, the knock-out or ablation of gene can lead the expression of the target gene below detectable levels or where with expression level is present at insignificant levels.

All publications and patent applications mentioned in the specification are indicative of the level of those skilled in the art to which this invention pertains. All publications and patent applications are herein incorporated by reference to the same extent as if each individual publication or patent application was specifically and individually indicated to be incorporated by reference.

Although the foregoing disclosure has been described in some detail by way of illustration and example for purposes of clarity of understanding, certain changes and modifications may be practiced within the scope of the appended claims.

Alzheimer's disease (AD) pathology includes neurofibrillary tangles rich in hyperphosphorylated tau protein. Pathological tau hyperphosphorylation is attributed to kinases including the widely-expressed GSK3 α and β.

Brains of individuals dying with Alzheimer's disease (AD) display neurofibrillary tangles (NFTs) rich in hyperphosphorylated tau protein and senile plaques rich in the Aβ fragment cleaved from the amyloid precursor protein (DeTure M A, Dickson D W. Mol Neurodegener. 2019;14(1):32; and Sery O, et al. Folia Neuropathol. 2013;51(1):1-9). Densities of NFTs in AD brains and levels of brain receptor type protein tyrosine phosphatase D (PTPRD) expression both display molecular genetic associations with variants in the gene encoding PTPRD (the receptor type protein tyrosine phosphatase D) (Chibnik L B, et al. Mol Psychiatry. 2018;23(6):1521-9; and Drgonova J, et al. Mol Med. 2015). There is specificity: densities of senile plaques are not significantly associated with PTPRD genomic variants (Chibnik L B, et al. Mol Psychiatry. 2018;23(6):1521-9).

Decreased PTPRD expression may reduce PTPRD's ability to downregulate principal tau hyperphosphorylating enzymes, glycogen synthase kinase 3 (GSK3) α and β (Cavallini A, et al. J Biol Chem. 2013;288(32):23331-47), via dephosphorylation of their activity-regulating (Hughes K, et al. EMBO J. 1993;12(2):803-8) phosphotyrosines 276 and 216, respectively (Henderson I M, et al. Biochem Pharmacol. 2022;202:115109). The prominent dietary flavonol quercetin provides positive allosteric modulation (PAM) of PTPRD's ability to dephosphorylate glycogen synthase kinase 3 (GSK3) α/β (Henderson I M, et al. Biochem Pharmacol. 2022;202:115109) and higher dietary intake of flavonols associated with decreased AD incidence, especially in individuals at higher genetic risk for AD (Holland T M, et al. Neurology. 2020; Shishtar E, et al. Am J Clin Nutr. 2020; and Jennings A, et al. JAMA Netw Open. 2024;7(9):e2434136) indirectly supports the role of PTPRD.

PTPRD regulates brain phosphorylation of GSK3 α and β pY279 and pY216. PTPRD is coexpressed in many of the neuronal subtypes that express GSK3 α and β. PAMs (positive allosteric modulators) of PTPRD's activity could reduce GSK3 activities with cell-type selectivity vs systemic inhibitors. PTPRD PAMs could inhibit tau hyperphosphorylation and slow development of neurofibrillary pathology. However, no selective PTPRD PAM with good drug-like properties has been reported. No current animal model tests the PTPRD-selectivity of influences of therapeutic candidates. Indeed, many current models require up to 2 years of aging to fully express relevant pathology.

An AD model, the 3xTg-AD mouse, contains variants that segregate independently of the chromosome 4 PTPRD locus: a chromosome 12 knock-in missense presenilin (PS1) M146V variant and a chromosome 2 insertion that overexpresses both the “Swedish” amyloid precursor protein (APP) K670_M671delinsNL APP and the tau (MAPT) P301L variants (Belfiore R, et al. Aging Cell. 2019;18(1):e12873). 3xTg-AD mice develop pathologically-hyperphosphorylated tau and Aβ deposits over a well-characterized 21-month time-course (Belfiore R, et al. Aging Cell. 2019;18(1):e12873). Administration of the abundant dietary flavonol quercetin to 3xTg-AD mice during the 3 (ip) or 12 (po) months prior to their sacrifice at 21 months reduces development of AD-like pathology (Paula PC, et al. Molecules. 2019;24(12); and Sabogal-Guaqueta A M, et al. Neuropharmacology. 2015;93:134-45).

Described herein are mouse models for AD pathophysiology and its PTPRD dependence. Disclosed herein are mouse models for AD pathophysiology and its dependence on PTPRD with 3xTg-AD variants on three genetic backgrounds: wild type, heterozygous PTPRD deletion (PTPRD+/−) and homozygous PTPRD deletion (PTPRD−/−).

The disclosed mouse models of AD can be used to study AD-like neurofibrillary pathology in 3xTg-AD mice on three genetic backgrounds: wild-type, heterozygous PTPRD deletion (PTPRD+/−) and homozygous PTPRD deletion (PTPRD−/−).

The disclosed mouse models can be used to identify compounds with improved drug-like properties in vitro, demonstrate their tolerability and improved bioavailability in vivo, document their in vivo anti-AD activities and show PTPRD-dependent activities. The disclosed mouse models of AD is a more efficient in vivo system for testing anti-AD activities of compounds (and the dependence of these anti-AD activities on PTPRD that can reduce AD neurofibrillary pathology.

Data described herein show that the comparison of results in the disclosed transgenic mice with the three genetic backgrounds can provide a in vivo system for more efficient testing of the of compounds and assessment of the dependence of their activities on PTPRD.

Described herein are the results of in vivo tests of lifelong genetic or postweaning (adolescent/adult) pharmacological modulation of PTPRD activity using 3xTg-AD mice including those with heterozygous PTPRD knockout, the potent irreversible PTPRD inhibitor pentilludin (Uhl G R. Front Psychiatry. 2023;14:1031283; and Henderson I M, et al. Biochem Pharmacol. 2022;195:114868), the PTPRD PAM quercetin (Henderson I M, et al. Biochem Pharmacol. 2022;202:115109) and antibodies that selectively recognize pathologically hyperphosphorylated tau (AT8) and Aβ.

### METHODS

Disclosed herein are transgenic mice and methods of producing the disclosed transgenic mice. Disclosed herein are non-human transgenic animal models useful for screening drugs or candidate drugs.

Disclosed herein are transgenic mice whose genome comprises: a) a knock-in first transgene encoding a mutant human presenilin-1 polypeptide having a mutation associated with Alzheimer's disease (AD) or AD-type pathology; b) a second transgene, encoding mutant human Tau protein having a mutation associated with AD or AD-type pathology, wherein the second transgene is operably linked to a promoter; and c) a third transgene, encoding human β-amyloid precursor protein (βAPP) having a mutation associated with AD or AD-type pathology, wherein the transgene is operably linked to a promoter; wherein at least one allele of the receptor type protein tyrosine phosphatase D (PTPRD) gene in the genome is ablated or deleted; and wherein expression of the transgenes results in a Tau or Aβ pathology in the transgenic mouse. In some aspects, the PTPRD gene can be “knocked out” (e.g., producing less PTPRD protein). A transgene can be used to transform a cell so that a genetic change can be present in the induced cell following incorporation of exogenous DNA. A permanent genetic change can be induced in a cell following incorporation of exogenous DNA, for example, into the genome of the cell. Vectors for stable integration include but are not limited to plasmids, retroviruses, other animal viruses, etc.

In some aspects, the disclosed transgenic mice can display neurofibrillary pathology or Tau or Aβ pathology and a reduced expression of PTPRD at 4 months of age. In some aspects, the disclosed transgenic mice can display AT8 immunoreactivity in hippocampal neuronal cells bodies and neuronal processes. In some aspects, only one allele of the PTPRD gene in the genome can be ablated (PTPRD+/−). In some aspects, both alleles of a receptor type protein tyrosine phosphatase D (PTPRD) gene in the genome are ablated (PTPRD−/−). In some aspects, the disclosed transgenic mice can display an increased amount of AT8 immunoreactivity in hippocampal neuronal cells bodies and neuronal processes at 4 months of age compared to a transgenic mouse that does not have a genome wherein at least one allele of receptor type protein tyrosine phosphatase D (PTPRD) gene is ablated or deleted. In some aspects, the disclosed transgenic mice can be hemizygous or homozygous for the human βAPP transgene; and wherein the mouse can be hemizygous or homozygous for the human tau transgene.

As used herein, the term “regulatory element” or “regulatory sequence” refers to promoters, promoter enhancers, internal ribosomal entry sites (IRES) and other elements that are capable of controlling expression (e.g., transcription termination signals, including but not limited to polyadenylation signals and poly-U sequences). Regulatory elements can direct constitutive expression. Regulatory element can also refer to enhancer elements. Specific gene specific promoters can be used. Such promoters allow cell specific expression or expression tied to specific pathways.

Transgenic knock-outs can have partial or complete loss of function in one or both alleles of an endogenous gene. The knock-out can be achieved by a variety of mechanisms, including introduction of a disruption of the coding sequence, deletion of the coding sequence. “Knock-outs” can also include conditional knock-outs, for example, where alteration of a target gene or target genes occurs upon exposure of the animal to a substance that promotes target gene alteration, introduction of an enzyme that promotes recombination at the target gene site or other method for directing the target gene alteration.

In general, a transgenic animal is produced by the integration of a given transgene into the genome in a manner that permits the expression of the transgene. Methods for producing transgenic animals are known in the art.

The expression levels of any of the genes or proteins described herein from the transgenic non-human animal or any cells derived thereof can be measured and subsequently compared to the expression level in a wild-type or otherwise healthy or normal non-human animal or cell or cell line.

Vectors can include plasmids, cosmids, and viruses (e.g., bacteriophage, animal viruses, and plant viruses), and artificial chromosomes (e.g., YACs). Vectors can comprise targeting molecules. A targeting molecule is one that directs the desired nucleic acid to a particular organ, tissue, cell, or other location in a subject's body. A vector, generally, brings about replication when it is associated with the proper control elements (e.g., a promoter, a stop codon, and a polyadenylation signal). Examples of vectors that are routinely used in the art include plasmids and viruses. The term “vector” includes expression vectors and refers to a vector containing a nucleic acid sequence coding for at least part of a gene product capable of being transcribed. A variety of ways can be used to introduce an expression vector into cells. In an aspect, the expression vector comprises a virus or an engineered vector derived from a viral genome. As used herein, “expression vector” is a vector that includes a regulatory region. A variety of host/expression vector combinations can be used to express the nucleic acid sequences disclosed herein. Examples of expression vectors include but are not limited to plasmids and viral vectors derived from, for example, bacteriophages, retroviruses (e.g., lentiviruses), and other viruses (e.g., adenoviruses, poxviruses, herpesviruses and adeno-associated viruses). Vectors and expression systems are commercially available and known to one skilled in the art.

A detectable marker or label can be introduced into the locus, where upregulation of expression can result in a detected change in the phenotype. Any of the vectors disclosed herein can also include a detectable marker or label. Such detectable labels can include but are limited to a tag sequence designed for detection (e.g., purification or localization) of an expressed polypeptide. Tag sequences include, for example, green fluorescent protein, glutathione S-transferase, polyhistidine, c-myc, hemagglutinin, or Flag™ tag, and can be fused with the encoded polypeptide and inserted anywhere within the polypeptide, including at either the carboxyl or amino terminus. The label can comprise any detectable moiety, including, for example, fluorescent labels, radioactive labels, and electronic labels.

In some aspects, the gene or genes described herein can be introduced into an appropriate vector for integration into the host. Examples of vectors include but are not limited to viruses.

In some aspects, a virus comprising Cre recombinase operably linked to any promoter can be can be used to carry out site specific recombination events.

Disclosed herein are cell lines or primary cell cultures. In some aspects, the cell lines or primary cell cultures can be derived from the transgenic mice described herein. In some aspects, cells or cell lines can be isolated from the transgenic mice described herein.

The cells lines described herein can be used for a variety of purposes including, but not limited to surveying human tissue and the like.

### METHODS OF SCREENING

Disclosed herein are methods of screening for a biologically active agents stimulate PTPRD activity in vivo. In some aspects, the methods can comprise: administering a candidate agent to a transgenic mouse disclosed herein, and determining the effect of said agent on the PTPRD activity. In some aspects, the methods can comprise determining the effect of said agent on the neurofibrillary pathology in mice with some remaining PTPRD activity and comparing the effects on the neurofibrillary pathology in mice with no remaining PTPRD activity.

Disclosed herein are methods of screening for biologically active agents that reduce neurofibrillary pathology in vivo. In some aspects, the methods can comprise: administering a candidate agent to a transgenic mouse disclosed herein, and determining the effect of said agent on the neurofibrillary pathology. In some aspects, the step of determining the effect of said on the neurofibrillary pathology can be assessed at 4 months of age.

Disclosed herein are methods of screening for biologically active agents that reduce AT8 immunoreactivity in vivo. In some aspects, the methods can comprise: administering a candidate agent to a transgenic mouse disclosed herein, and determining the effect of said agent on the AT8 immunoreactivity. In some aspects, the step of determining the effect of said on the AT8 immunoreactivity can be assessed at 4 months of age.

The methods described herein can provide a more convenient way to test of candidate biologically active agents that can reduce neurofibrillary pathology and AT8 immunoreactivity, The transgenic mice with heterozygous or homozygous PTPRD deletion described herein can be dosed (e.g., administered) with the candidate biologically active agent or vehicle from weaning until sacrifice at 4 months of age. Brain levels of AD neurofibrillary and AT8 immunoreactivity can then be assessed in brains of each animal, and levels of this neurofibrillary or AT8 pathology can be compared in mice treated with the candidate biologically active agent to those treated with vehicle.

The methods described herein can provide a test of whether a candidate biologically active agent works by engaging PTPRD. For example, the effects of the candidate biologically active agent can be assessed on the development of AT8 of neurofibrillary pathology and compared to the transgenic mice disclosed herein with heterozygous PTPRD knockout vs the transgenic mice disclosed herein with homozygous knockout both treated with the candidate biologically active agent. If the candidate biologically active agent (dosed between weaning and 4 months of age) reduces the AT8 and neurofibrillary pathology in transgenic mice disclosed herein with heterozygous PTPRD knockout but fails to reduce AT8 and neurofibrillary pathology in transgenic mice disclosed herein with the homozygous PTPRD knockout, the result provides presumptive evidence that the mechanism of the candidate biologically active agent involves PTPRD.

Also, disclosed herein are methods for identifying biologically active agents or compounds (e.g., peptides, cyclic peptides, peptidomimetics, small molecules, small organic molecules, or other drugs). Agents or compounds identified as described herein can be used in an animal model to determine the mechanism of action, efficacy, toxicity or side effects of treatment with said agents or compounds.

Test compounds can be formulated into pharmaceutical compositions by admixture with pharmaceutically acceptable non-toxic excipients or carriers and administered to transgenic non-human animals described herein by any route of administration. For example, parenteral routes such as subcutaneous, intramuscular, intravascular, intradermal, intranasal, inhalation, intrathecal, or intraperitoneal administration, and enteral routes such as sublingual, oral, or rectal administration can be used.

### EXAMPLES

### Example 1: Enhancing PTPRD Positive Allosteric Modulators and Mouse Models for Alzheimer's Disease Neurofibrillary Pathology

3xTg-AD mice provide a useful model for AD pathology (Javonillo, et al. Front. Neurosci. 15:785276 (2022). Aged 3xTg-AD mice display abundant neurofibrillary pathology that can be readily detected with AT8 antibodies (as well as those that recognize other AD-related epitopes) (Javonillo, et al. Front. Neurosci. 15:785276 (2022). The time-course of evolution of AD-like pathologies and mnemonic deficits has been elucidated in these mice (Javonillo, et al. Front. Neurosci. 15:785276 (2022).

Establishment of reliable assays of drug effects in younger animals will provide a major benefit for AD research. Such assays can be useful in developing and identifying anti-AD strategies and therapies.

Establishment of reliable assays that would document the PTPRD dependence of benefits will also provide value. The effects of compounds on PTPRD and their actions in AD are limited by currently-available animal models. AD and neurofibrillary animal models and the use of the same can be used to determine the in vivo benefit of compounds that depend on actions of PTPRD.

Production and aging of 3xTg-AD mice with PTPRD+/+, PTPRD+/− and PTPRD−/− genetic backgrounds. The viability and fertility of 3xTg-AD mice (JAX (004807 B6; 129-Tg (APPSwe, tauP301L) 1Lfa Psen1^{tm1Mpm}/Mmjax) with reduced expression of PTPRD has been documented. Breeding 3xTg-AD/PTPRD+/− males and females yields 3xTg-AD mice with each of the three PTPRD genotypes (termed “genetic backgrounds” herein). Fewer 3xTg-AD mice with PTPRD−/− backgrounds are born than expected by Mendelian ratios (PTPRD+/−×PTPRD+/− crosses also produce PTPRD−/− mice at rates lower than expected by Mendelian ratios).

Some mice of both genders with the possible genotypes survive to age 21 months. There is excess mortality of aging male 3xTg-AD mice with any PTPRD genotype and among the PTPRD−/− homozygous knockouts.

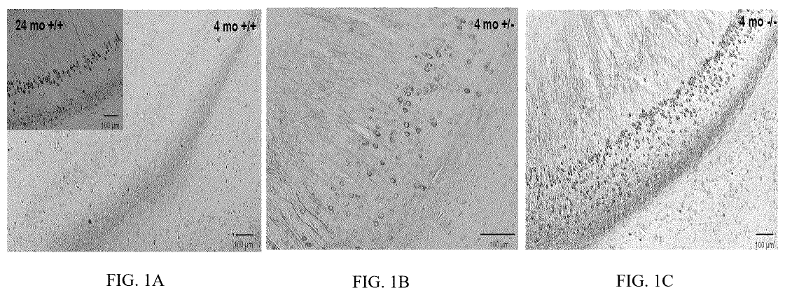

Phosphotau immunoreactivity in brains of 3xTg-AD mice with PTPRD+/+, PTPRD +/− and PTPRD−/− genetic backgrounds (n=2/genotype): AT8 immunoreactivity (pathologically phosphorylated tau) was assessed at 4 months of age (Belfiore, R., et al., Temporal and regional progression of Alzheimer's disease-like pathology in 3xTg-AD mice. Aging Cell, 2019. 18(1): p. e12873). Remarkably, the results show abundant AT8 immunoreactivity in hippocampal neuronal cell bodies and neuronal processes in 4 month-old mice 3xTg-AD mice with PTPRD−/− genetic background (e.g., lacking PTPRD; FIG. 1, R panel). No such immunoreactivity was observed in 4 month old 3xTg-AD mice with wild type PTPRD+/+ backgrounds (FIG. 5, L panel). 3xTg-AD mice with PTPRD+/− genetic background (e.g., expressing half of wild type levels of PTPRD) display substantial hippocampal AT8 immunoreactivity (FIG. 5, middle panel) though less intense staining than in 3xTg/PTPRD−/− mice. Counts of immunoreactive neurons/half brain section averaged 190, 61 and 0 for −/−, +/− and +/+PTPRD genotypes, respectively. AT8 immunoreactivity in 4 month-old 3xTg-AD mice with no PTPRD was almost as abundant as that in 24 month old 3xTg-AD mice with wild type PTPRD (232 neurons/section; FIG. 1, R panel vs inset). These combined initial data for fertility, viability and earlier development of pathologically-phosphorylated tau with reduced PTPRD expression in 3xTg-AD mice demonstrate the usefulness of the mouse model for pathological tau in AD.

3xTg-AD mice heterozygous for PTPRD (+/−) will be bred and housed in the VA Maryland Healthcare System AALAC-certified animal facility and genotyped (Belfiore, R., et al., Temporal and regional progression of Alzheimer's disease-like pathology in 3xTg-AD mice. Aging Cell, 2019. 18(1): p. e12873; and Drgonova, J., et al., Mouse model for PTPRD associations with WED/RLS and addiction: reduced expression alters locomotion, sleep behaviors and cocaine-conditioned place preference. Mol Med, 2015). At least an n of 8 will be produced.

Seven μM sections from paraffin-embedded brains from mice which are anesthetized and perfused with 4% depolymerized paraformaldehyde will be assessed. Sections are heated to 58° C. for 1 hr, cooled, deparaffined in xylene, rehydrated using graded alcohols, incubated in microwaved/boiled 10 mM sodium citrate, pH 6.0, incubated in 3% H₂O₂ and blocked with M.O.M. Mouse IgG blocker. Sections are incubated overnight at 4° C. with AT8 antibody (MN1020, 1.25 μg/mL), washed, incubated with M.O.M. biotinylated anti-mouse IgG (1:250), incubated with avidin-biotin complex, and washed and developed with diaminobenzidine using avidin-biotin horseradish peroxidase (Vector Labs). Phosphotau immunoreactive hippocampal cell counts are quantitated by an investigator blinded to age, genotype and treatment using a Zeiss AxioImager M2 (40× objective) ((Belfiore, R., et al., Temporal and regional progression of Alzheimer's disease-like pathology in 3xTg-AD mice. Aging Cell, 2019. 18(1): p. e12873).

Other sections of the brain will be evaluated including but not limited to the entorhinal cortex; and expression of other epitopes relevant to AD phosphotau and amyloid will be assessed ((Belfiore, R., et al., Temporal and regional progression of Alzheimer's disease-like pathology in 3xTg-AD mice. Aging Cell, 2019. 18(1): p. e12873).

The mouse model described herein can be used as an invaluable test bed for compounds that target the PTPRD-related mechanisms that impact neurofibrillary pathology in AD. This model can also provide a more convenient way to test ono-PTPRD interventions. Animals with the 3xTg-AD variants combined with PTPRD+/−reduction model common human allelic variants that provide 50-60% individual differences in levels of PTPRD expression in human postmortem brains (Drgonova, J., et al., Mouse model for PTPRD associations with WED/RLS and addiction: reduced expression alters locomotion, sleep behaviors and cocaine-conditioned place preference. Mol Med, 2015). Expression of pathologically hyperphosphorylated AT8-immunoreactive tau in these mice at 4 months of age, a time when there is no such immunoreactivity in 3xTg-AD mice with wild type PTPRD background, provides a test bed on which to validate any tau-related approach. In some aspects, these mice can be called 4xTg-AD, since they combine a) overexpression of rare tau and amyloid precursor protein variants that are found uncommonly in Alzheimer's disease-related dementias with Mendelian transmission, b) knockout of presenilin with c) a model for common human PTPRD variation (Drgonova, J., et al., Mouse model for PTPRD associations with WED/RLS and addiction: reduced expression alters locomotion, sleep behaviors and cocaine-conditioned place preference. Mol Med, 2015) that is associated with individual differences in NFT densities (Chibnik, L. B., et al., Susceptibility to neurofibrillary tangles: role of the PTPRD locus and limited pleiotropy with other neuropathologies. Mol Psychiatry, 2018. 23(6): p. 1521-1529).

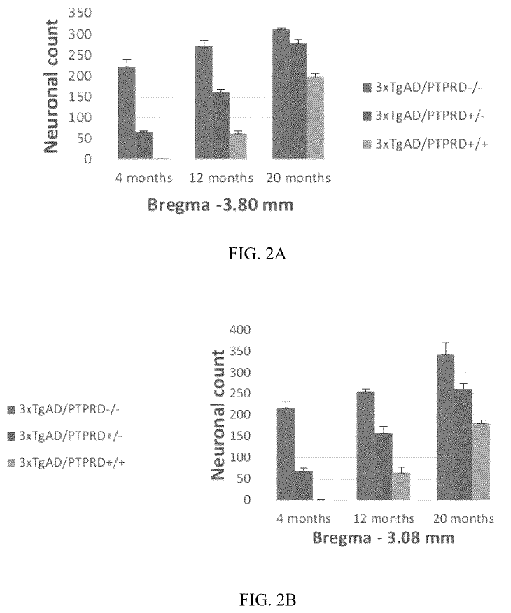

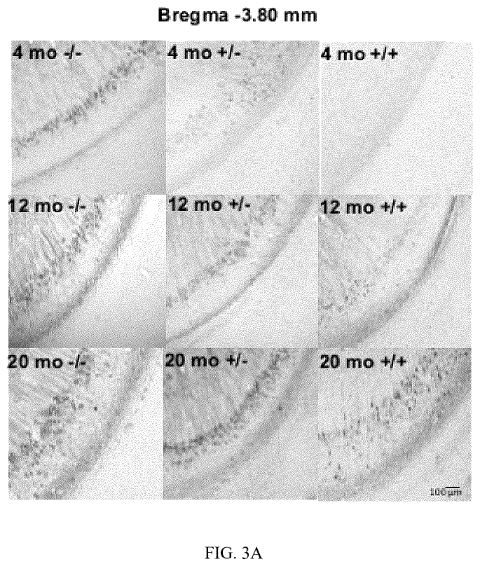

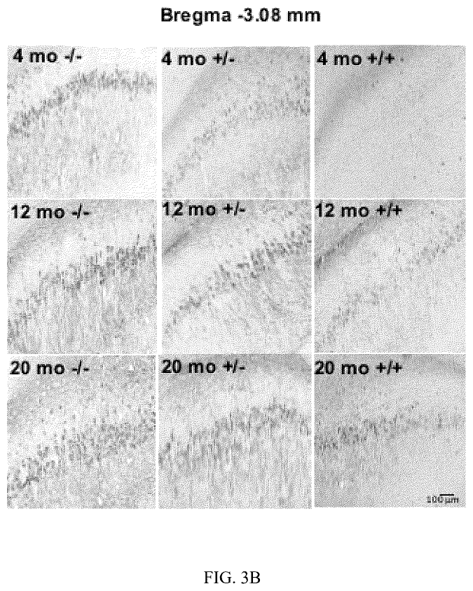

The data disclosed herein is sufficiently robust such that these findings can be extended in additional 4 month-old 3xTg-AD mice with different genetic backgrounds: no hippocampal phosphotau AT8 immunoreactivity in 3xTg-AD/PTPRD+/+ wild type mice, substantial immunoreactivity in 3xTg-AD/PTPRD+/− heterozygous knockout mice and dense immunoreactivity in 3xTg-AD/PTPRD−/− homozygous knockout mice. FIG. 2 shows the neuronal count in 3xTg-AD mice with +/+, +/−, and −/−PTPRD−/−. FIG. 3 shows AT-8 immunoreactive hyperphosphorylated tau in 3xTg-AD mice with +/+, +/−, and −/−PTPRD−/− at 4 months, 12 months, and 20 months.

### Example 2: Receptor Type Protein Tyrosine Phosphatase D Contributes to Alzheimer's Disease Like Neurofibrillary Pathology: Genetic and Pharmacologic Support

Variants in the human receptor type protein tyrosine phosphatase D (PTPRD) gene are associated with levels of brain PTPRD mRNA and with densities of neurofibrillary tangle (NFT) pathology in Alzheimer's disease (AD) brains, though not with densities of Aβ-rich senile plaques. NFTs are largely composed of tau protein that is hyperphosphorylated by serine/threonine kinases that prominently include glycogen synthase kinases (GSK)3 α and β.

Tyrosine phosphorylation of GSK3 α and β enhances their kinase activities. Phosphotyrosine GSK3s are briskly dephosphorylated by PTPRD, providing a plausible molecular mechanism for genetic associations with NFT densities. However, there has been neither animal model validation of these human genetic observations nor testing of the effects of pharmacological PTPRD modulation post-weaning. Described herein is the creation of 3xTg-AD/PTPRD+/− mice that are viable and tolerate week 6-16 alternating day (qod) administration of 25 mg/kg of the PTPRD positive allosteric modulator (PAM) quercetin. 3xTg-AD mice tolerate week 6-16 qod administration of 100 mg/kg of the PTPRD inhibitor pentilludin. Hippocampal neurons from 16 week-old 3xTg-AD/PTPRD+/− mice and those from pentilludin-treated 3xTg-AD mice display hyperphosphorylated tau at levels not seen in vehicle-treated 3xTg-AD mice or pentilludin-treated wildtype mice. By contrast, fewer hippocampal neurons display hyperphosphorylated tau in quercetin-treated 16 week-old 3xTg-AD/PTPRD+/− mice. 3xTg-AD/PTPRD+/− mice do not reliably develop Aβ plaque-like structures until 20 months of age. These results demonstrate the PTPRD roles in AD neurofibrillary pathophysiology and reducing AD pathology using improved PTPRD PAMs.

PTPRD is highly expressed by neurons, regulates brain phosphorylation of important activity-regulating GSK3 α and β phosphotyrosines pY279 and pY216, respectively. Genome wide association results link PTPRD variants to individual differences in neurofibrillary tangle densities in AD brains and results from dietary records of aging individuals who go on to develop AD suggest that modulating PTPRD's activity may be effective in reducing the activity of GSK3. However, no selective PTPRD PAMs have been developed as pharmacotherapeutics. No animal model allows testing of the PTPRD-selectivity of influences of therapeutic candidates. Current animal models of AD require many months of aging to express relevant pathology.

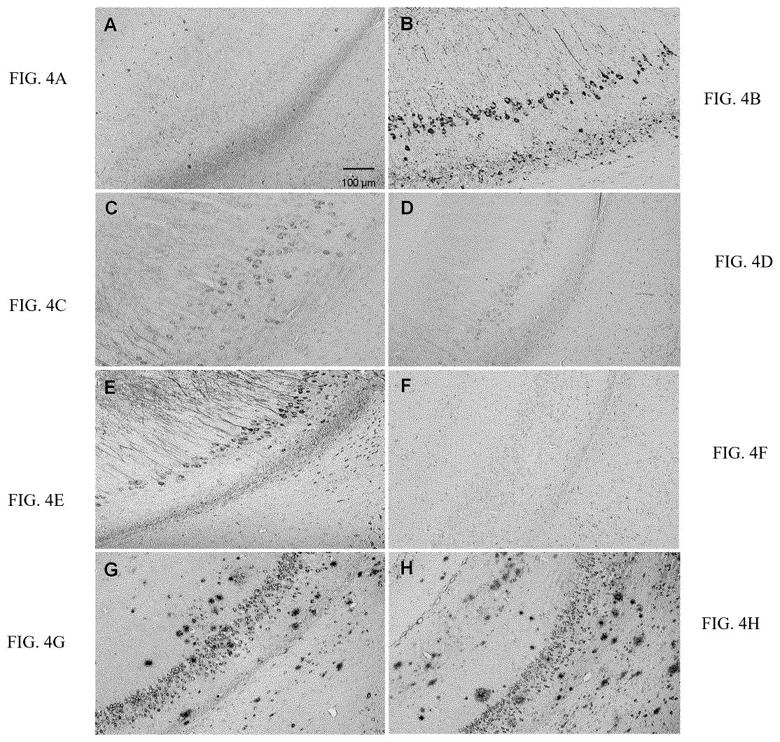

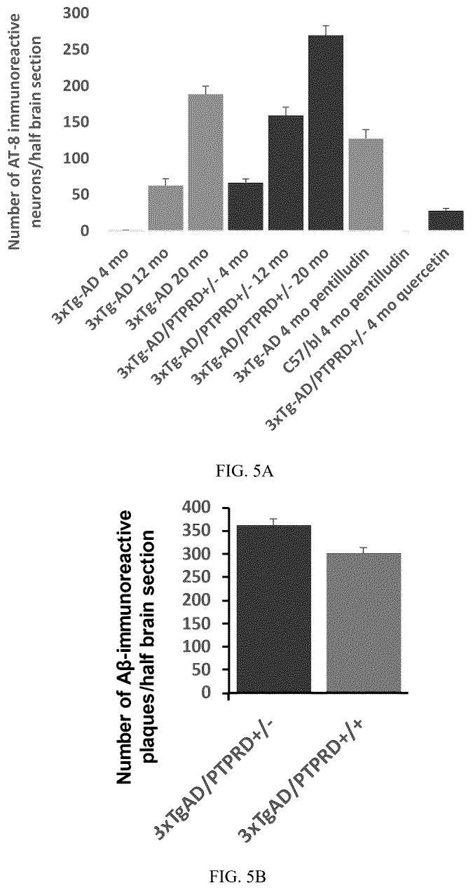

20 month old 3xTg-AD mice with wildtype PTPRD display AT8-immunoreactive, pathologically-hyperphosphorylated tau that fills much of the perikarya of hippocampal neurons in sections taken at −3.08 and −3.8 mm from bregma(Belfiore R, et al. Aging Cell. 2019; 18(1):e12873) (FIG. 4B). There is one AT8-immunoreactive neuron (averaged in half brain sections from −3.08 and −3.8 mm from bregma) in 3xTg-AD mice sacrificed at 4 months, 63 at 12 months and 188 at 20 months(Belfiore R, et al. Aging Cell. 2019;18(1):e12873); FIG. 5). By contrast, the 20 month-old 3xTg-AD and 3xTg-AD/PTPRD+/− mice reliably display Aβ-immunoreactive senile-plaque structures (FIGS. 1G, 1H).

3xTg-AD/PTPRD+/− mice are fertile; most survive to 20 months of age. These mice display 66 AT8 immunoreactive hippocampal neurons at 4 months (n=6), 160 at 12 months and 270 at 20 months.

There are effects of pharmacologic reductions in PTPRD activity using pentilludin (100 mg/kg po, alternating days (qod) weeks 6-16). Pentilludin-treated 3xTg-AD mice display 127 AT8-immunoreactive hippocampal at 4 months of age, while vehicle-treated mice display 6 (n=6, ANOVA p=2×10⁻¹⁰). There are no AT8 immunoreactive neurons in 4 month-old pentilludin-treated wildtype C57bl mice (n=6, p=2×10⁻¹⁰ vs pentilludin effects in 3xTg-AD mice).

There are contrasting effects of quercetin, the positive allosteric modulator of PTPRD's ability to dephosphorylate and downregulate GSK3s (Henderson I M, et al. Biochem Pharmacol. 2022;202:115109). 25 mg/kg quercetin ip qod from weeks 6-16 reduces the numbers of AT8 immunoreactive hippocampal neurons in 4 month-old 3xTg-AD/PTPRD+/− mice to 27 (n=5) vs the 64 AT8-positive neurons found in vehicle-treated 3xTg-AD/PTPRD+/− mice (n=6, p=8×10⁻⁹).

There are different effects of altering PTPRD on deposition of the prominent amyloid constituent, Aβ. No Aβ-immunoreactive elements are noted in hippocampus or overlying cortex of 4 month-old 3 xTg-AD mice with any PTPRD genotype or with pentilludin treatment. Few are seen at 12 months. At 20 months, 3xTg-AD mice with wildtype PTPRD (n=4) display 301 Aβ-immunoreactive plaque like structures/half brain section of hippocampus and overlying cortex while 3xTg-AD/PTPRD+/− mice display 361 (n=3, p=5×10⁻⁴) FIG. 5B).

Bromination at quercetin's 6 position provided consistently-improved PAM activity vs quercetin. There was a sharp structure activity relationship at this position. 6-iodo quercetin provided a little less PAM activity, and much less was provided by 6-chloro or 6-hydroxyl substitutions. There was no PAM activity with 6-methyl substitution. 6, 8 dibromoquercetin inhibited PTPRD's phosphatase. 6 bromo-quercetin (6BrQ) provided almost twice the PTPRD PAM activity provided by quercetin. This PAM activity was also greater than that of every 8-position substitution tested. Quercetin benefits for AD have been attributed to a number of mechanisms, most often to its antioxidant properties. 6BrQ retains quercetin's antioxidant properties as assessed by cyclic voltammetry.

In vivo studies identify 6BrQ >quercetin reductions in AT8 immunoreactive neuronal phosphorylated tau accumulation in 3xTg-AD/PTPRD+/− mice. To seek in vivo correlates of the in vitro structure-activity studies, quercetin, 6BrQ or vehicle (25 mg/kg/alternate day, ip, weeks 6-16) were administered to 3xTg-AD/PTPRD+/− mice. Quercetin treatment reduced the accumulation of hyperphosphorylated tau in hippocampal neurons in these mice. There were 29 AT8 immunoreactive hippocampal neurons in quercetin-treated 4 month-old 3xTg-AD/PTPRD +/− mice vs the 69 AT8-positive neurons found in vehicle-treated 3xTg-AD/PTPRD+/− mice (p=8×10⁻⁹).

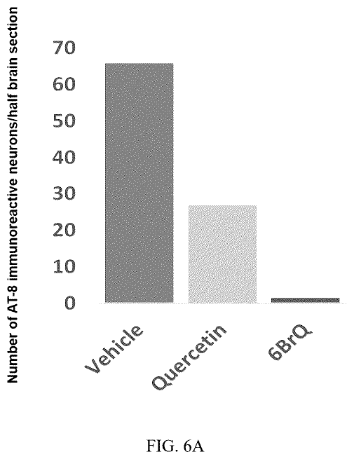

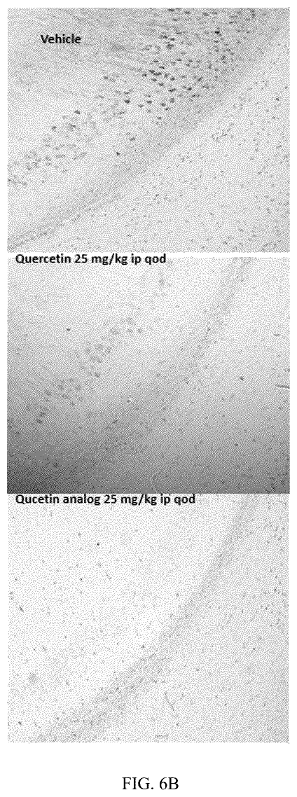

6BrQ provided a remarkably-greater effect than quercetin. There were no AT8 immunoreactive neurons in some treated mice and one in others; the mean was 0.3. These improved results were significantly-different from those obtained in either vehicle or quercetin-treated 3xTg-AD/PTPRD+/− mice (Tukey-Kramer q=21 and 8.1, respectively vs critical value 4.69). For example, FIG. 6 also shows AT8 immunoreactivity in hippocampal neurons after treatment with vehicle, quercetin or 6BrQ.

The findings that adding reduced PTPRD expression hastens development of neurofibrillary pathology in 3xTg-AD mice fit well with human genetic associations (Chibnik L B, et al. Mol Psychiatry. 2018;23(6):1521-9; and Drgonova J, et al. Mol Med. 2015). Lifelong differences in levels of PTPRD expression thus alter development of neurofibrillary pathology in 3xTg-AD mice and, likely, in humans. There is specificity: 3xTg-AD mice with reduced PTPRD expression develop Aβ-immunoreactive senile-plaque-like pathology at about the same time as 3xTg-AD mice with wildtype PTPRD, consistent with the lack of a strong association between human PTPRD variation and senile plaque densities in AD brains (Chibnik L B, et al. Mol Psychiatry. 2018;23(6):1521-9).

Results of pharmacological studies with the potent, irreversible PTPRD inhibitor pentilludin (Uhl G R. Front Psychiatry. 2023;14:1031283; and Henderson I M, et al. Biochem Pharmacol. 2022;195:114868) support these genetic results. Alternating day, week 6-16, 100 mg/kg pentilludin result in even more AT-8 immunoreactive neurons in 3xTg-AD mice than the heterozygous PTPRD knockout.

Quercetin is a contrasting pharmacologic tool, since it increases PTPRD's abilities to dephosphorylate and thus downregulate activities of GSK3α and GSK3β (Henderson I M, et al. Biochem Pharmacol. 2022; 202:115109). Quercetin administration for weeks 6-16 reduces development of neurofibrillary pathology in 3xTg-AD/PTPRD+/− mice by almost half. These results add to prior observations, noted above, concerning AD-related benefits of quercetin in 3xTg-AD mice (Paula P C, et al. Molecules. 2019; 24(12); and Sabogal-Guaqueta A M, et al. Neuropharmacology. 2015;93:134-45) and of flavonols in humans, especially those who display higher genetic risk for AD and low baseline flavonol intake (Holland T M, et al. Neurology. 2020; Shishtar E, et al. Am J Clin Nutr. 2020; and Jennings A, et al. JAMA Netw Open. 2024; 7(9):e2434136). Quercetin also has other actions of possible relevance for AD, including antioxidant and anti-inflammatory actions as well as inhibition of acetylcholinesterase and Aβ aggregation (Sabarathinam S. Sci Rep. 2024; 14(1):14852).

Combined mouse and human genetic and pharmacologic results, taken together, thus support sizable roles for PTPRD in AD neurofibrillary pathophysiology. These results demonstrate that improved PTPRD PAMs, administered during adulthood, can reduce human AD pathophysiology, especially in those at enhanced genetic risk of developing AD.

In sum, the results demonstrate that treatments from 6 weeks of age to 16 weeks of age alter pathology in 3xTg-AD PTPRD+/− and in 3xTg AD mice with wildtype PTPRD. For example, quercetin treatment (25 mg/kg) weeks 6-16 reduces neurofibrillary pathology in the 3xTg-AD/PTPRD+/− mice by about half. The results confirm that treatment of 3xTg-AD mice (wildtype PTPRD) with pentilludin (inhibitor) increases neurofibrillary pathology in mice at 4 months of age (e.g., a result that can replicate the effect of the lifelong knockout with treatment as young adult to adulthood). Further, the results show that the neurons that express neurofibrillary pathology in the pentilludin-treated mice also express PTPRD (double staining immunohistochemistry). Second, plaque-like Aβ immunoreactivity does not develop until later in the 3xTg-AD/PTPR +/− mice. At 20 mos, they do have statistically greater amounts, but at 12 mos neither 3xTg-AD nor 3xTg-AD/PTPRD+/− mice have any Aβ staining.

Materials and Methods. 3xTg-AD mice (JAX (004807 B6; 129-Tg (APPSwe, tauP301L)1Lfa Psen1tm1Mpm/Mmjax) were crossed with heterozygous PTPRD knockout mice. Pups were genotyped (Drgonova J, et al. Mol Med. 2015; and Belfiore R, et al. Aging Cell. 2019; 18(1): e12873). Mice were treated with PEG600 vehicle, quercetin (25 mg/kg ip every other day (qod)) or pentilludin (NetChem, >92% pure; 100 mg/kg po (gavage) qod from week 6 of age until sacrifice at 16 weeks. Mice were then sacrificed by perfusion with buffered 4% depolymerized paraformaldehyde under anesthesia. 7 μM coronal sections from paraffin-embedded brains (levels −3.08 and −3.8 mm from bregma) were stained by heating to 58° C. for 1 hr, cooling, deparaffining in xylene, rehydrating with graded alcohols, incubations in 10 mM sodium citrate, pH 6.0, 3% H2O2, mouse IgG blocker and AT8 antibody (Thermo Fisher MN1020, 1.25 μg/mL) or anti-Aβ antibody (Sigma MABN10, 1:2000 dilution) overnight at 4° C. followed by washing, incubations with M.O.M. biotinylated anti-mouse IgG (1:250) or anti-RRR IgG, avidin-biotin complex/horseradish peroxidase (Vector) and development with diaminobenzidine. Numbers of phosphotau/AT8-immunoreactive hippocampal neurons and of Aβ-immunoreactive elements in hippocampus and overlying cortex were quantitated in half brain stitches obtained with automated slide scanning via a Zeiss AxioImager M2 with a 10×0.3 NA objective with analysis using ImageJ by an investigator blinded to genotype and treatment. Results presented average neuronal counts at −3.08 and −3.8 mm from bregma.

What is claimed is:

1. A transgenic mouse whose genome comprises:
a) a knock-in first transgene encoding a mutant human presenilin-1 polypeptide having a mutation associated with Alzheimer's disease (AD) or AD-type pathology;
b) a second transgene, encoding mutant human Tau protein having a mutation associated with AD or AD-type pathology, wherein the second transgene is operably linked to a promoter;
c) a third transgene, encoding human β-amyloid precursor protein (βAPP) having a mutation associated with AD or AD-type pathology, wherein the transgene is operably linked to a promoter; and
wherein at least one allele of the receptor type protein tyrosine phosphatase D (PTPRD) gene in the genome is ablated or deleted, and
wherein expression of the transgenes results in a Tau or Aβ pathology in the transgenic mouse.

2. The transgenic mouse of claim 1, wherein the transgenic mouse displays neurofibrillary pathology or Tau or Aβ pathology and a reduced expression of PTPRD at 4 months of age.

3. The transgenic mouse of claim 2, wherein the transgenic mouse displays AT8 immunoreactivity in hippocampal neuronal cells bodies and neuronal processes.

4. The transgenic mouse of claim 2, wherein only one allele of the PTPRD gene in the genome is ablated (PTPRD+/−).

5. The transgenic mouse of claim 1, wherein both alleles of a receptor type protein tyrosine phosphatase D (PTPRD) gene in the genome are ablated (PTPRD−/−).

6. The transgenic mouse of claim 1, wherein the transgenic mouse displays an increased amount of AT8 immunoreactivity in hippocampal neuronal cells bodies and neuronal processes at 4 months of age compared to a transgenic mouse that does not have a genome wherein at least one allele of receptor type protein tyrosine phosphatase D (PTPRD) gene is ablated or deleted.

7. The transgenic mouse of claim 1, wherein the mouse is hemizygous or homozygous for the human βAPP transgene; and wherein the mouse is hemizygous or homozygous for the human tau transgene.

8. A cell line or primary cell culture derived from the transgenic mouse according to claim 1.

9. A method of screening for biologically active agents that stimulate PTPRD activity in vivo, the method comprising: administering a candidate agent to the transgenic mouse of claim 1, and determining the effect of said agent on the PTPRD activity.

10. A method of screening for biologically active agents that reduce neurofibrillary pathology in vivo, the method comprising: administering a candidate agent to the transgenic mouse of claim 1, and determining the effect of said agent on the neurofibrillary pathology.

11. The method of claim 10, wherein the step of determining the effect of said agent on the neurofibrillary pathology is assessed at 4 months of age.

12. A method of screening for biologically active agents that reduce AT8 immunoreactivity in vivo, the method comprising: administering a candidate agent to the transgenic mouse of claim 1, and determining the effect of said agent on the AT8 immunoreactivity.
